(19) United States
(12) Patent Application Publication
(10) Pub. No.: US 2026/0144816 A1
(43) Pub. Date: May 28, 2026

(54) COMPOSITIONS AND METHODS FOR TREATING DEMENTIA

(71) Applicant: Board of Regents, The University of Texas System, Austin, TX (US)

(72) Inventor: LUCA CICALESE, Galveston, TX (US)

(21) Appl. No.: 19/355,527

(22) Filed: Oct. 10, 2025

Related U.S. Application Data
(60) Provisional application No. 63/706,775, filed on Oct. 14, 2024.

(51) Int. Cl. A61K 33/06; A61K 31/436; A61K 31/517; A61K 31/522; A61K 31/5377; A61K 31/573; A61K 31/662; A61K 31/675; A61K 31/7056; A61K 38/13; C07K 16/28

(52) U.S. Cl. CPC A61K 33/06; A61K 31/436; A61K 31/517; A61K 31/522; A61K 31/5377; A61K 31/573; A61K 31/662; A61K 31/675; A61K 31/7056; A61K 38/13; C07K 16/2866

(57) ABSTRACT

Aspects are directed to providing or administering novel therapeutic combinations for the treatment of tauopathies such as AD and other neurological deficiencies or dementias. Certain aspects include methods of using two or more drugs selected from an immunomodulator (IMD), antiviral (AVT), and magnesium (Mg) for the treatment of AD and/or other tauopathies; or compositions containing two or more drugs selected from an immunomodulator (IMD), antiviral (AVT), and magnesium (Mg).

### PRIORITY PARAGRAPH

This Application claims priority to US Provisional Application 63/706,775 filed Oct. 14, 2024 which is incorporated herein by reference in its entirety.

### STATEMENT REGARDING FEDERALLY FUNDED RESEARCH

None.

### FIELD

Embodiments are directed generally to the field of neuroscience and medicine. In particular, embodiments are directed to methods for treating dementia.

### BACKGROUND

Alzheimer's disease (AD) is characterized by two hallmark lesions in the central nervous system (CNS): neurofibrillary tangles of tau protein and deposits of aggregated amyloid beta (AB). However, the disruption of synaptic spines, likely caused by the detrimental binding of small Aß oligomers, triggers cognitive decline. Synaptic failure is widely recognized as one of the initial events driving symptomatic AD, making it a prime target for therapeutic intervention. Unfortunately, a successful pharmacological strategy to address this issue remains elusive.

Beyond these primary factors, several other elements play critical roles in AD. Neurodegeneration and Aß accumulation activate microglia, leading to the secretion of proteolytic enzymes needed to phagocytose Aß peptides and the release of pro-inflammatory cytokines. Consequently, inflammation and a complex array of immunologic reactions occur in the affected brain areas, exacerbating AD pathology. Contrary to the traditional view of the brain as an “immune privileged” site, research has shown otherwise. In 1948, Peter Medawar, a pioneer in transplantation, discovered that grafts transplanted into the brains of animals were rejected by peripheral immune cells, indicating that activated lymphocytes can cross the blood-brain barrier (BBB). This finding has since been validated by other researchers.

The U.S. population afflicted by AD is projected to reach 15 million by 2050. The societal cost of long-term care for these patients, who inevitably lose their self-sufficiency, will be virtually unsustainable, as no cure for AD currently exists. Therefore, the need to develop innovative, effective treatments is urgent. However, due to the multifactorial nature of AD, use of single therapeutic target or agent has thus far proven ineffective in producing a curative treatment in humans. With these promising insights, the Inventor has set out to provide compositions and methods to address the need for additional treatments for dementia and AD.

### SUMMARY

Aspects of the invention provide a solution to the problems discussed above by providing novel therapeutic combinations for the treatment of dementia, tauopathies such as AD, and other neurological deficiencies. A novel approach aimed at simultaneously addressing various targets is presented. In certain aspects, FDA-approved drugs can be repurposed for treating subjects at risk of dementia. In certain aspects, an antiviral can be combined with magnesium to treat dementia patients. The antiviral can be valganciclovir, administered with or without magnesium, to dementia patients or subjects at risk of developing dementia. In certain aspects, different immunomodulators (IMDs) can be combined to treat dementia or AD. The IMDs can include tacrolimus, mycophenolate, rapamycin, or belatacept. In specific aspects, the treated subject has not undergone an organ transplant. In specific aspects, a dementia patient is not taking immunosuppressive drugs when a treatment described herein is administered.

Embodiments are directed to methods of using two or more drugs selected from an immunomodulator (IMD), antiviral (AVT), and magnesium (Mg) for the treatment of AD and/or other tauopathies.

Certain embodiments are directed to methods of treating a tauopathy or neurodegenerative disease comprising administering a therapeutic amount of two or more drugs selected from an immunomodulator (IMD), antiviral (AVT), and magnesium (Mg) to a subject having or at risk of developing a tauopathy or neurodegenerative disease. The dose can range from about 0.5, 1, 5, 10, 25, 50, 100, 150, 200, 250, 300, 350, 400, 450, 500, 550, 600, 650, 700, 750, 800, 850, 900, 950, to 1000 mg or more. The tauopathy or neurodegenerative disease can be Alzheimer's disease or dementia. In certain aspects, the two or more drugs selected from an immunomodulator (IMD), antiviral (AVT), and magnesium (Mg) are administered at a dose of 200 mg to 500 mg daily for each component. In certain aspects, the two or more drugs selected from an immunomodulator (IMD), antiviral (AVT), and magnesium (Mg) are administered at a dose greater than 300 mg daily for each component. The method can include administering the two or more drugs selected from an immunomodulator (IMD), antiviral (AVT), and magnesium (Mg) chronically, or for 10 to 30 days, 2 to 5 weeks, 1 to 6 months, or 1 to 20 years. The therapeutic composition can be administered by oral administration. A tablet, capsule, pill, powder, sustained-release formulation, solution, or suspension containing the two or more drugs selected from an immunomodulator (IMD), antiviral (AVT), and magnesium (Mg) can be administered orally. In certain aspects, the combination therapy is formulated into a single dosage form, i.e., co-formulated.

Certain embodiments are directed to methods for treating dementia comprising administering a combination therapy comprising a dose of two or more drugs selected from an immunomodulator (IMD), antiviral (AVT), and magnesium (Mg) to a subject having or at risk of developing dementia, wherein the dose of each drug is about 0.5 mg to 1000 mg. In certain aspects, the subject has or is at risk of developing Alzheimer's disease. The IMD can be one or more of cyclosporine, tacrolimus, sirolimus (rapamycin), everolimus, mycophenolate mofetil (MMF), azathioprine, prednisone, basiliximab, thymoglobulin, or belatacept. The AVT can be one or more of valganciclovir, ganciclovir, foscarnet, cidofovir, acyclovir, valacyclovir, famciclovir, letermovir, maribavir, or brincidofovir. In certain aspects, the IMD or AVT is administered at a dose of 0.5 mg to 500 mg daily. The combination therapy can be administered for 10 to 30 days, 2 to 5 weeks, or 1 to 6 months. In certain aspects, the combination therapy is administered by oral or intranasal administration. Oral administration comprises a tablet, capsule, pill, powder, sustained-release formulation, solution, or suspension. In certain aspects, the immunomodulator (IMD), antiviral (AVT), and magnesium (Mg) are co-formulated. The immunomodulator (IMD), antiviral (AVT), and magnesium (Mg) can be administered within 10 to 60 minutes of each other.

Certain embodiments are directed to compositions comprising a co-formulation of an immunomodulator (IMD) and an antiviral (AVT). The composition can further include magnesium. The IMD can be one or more of cyclosporine, tacrolimus, sirolimus (rapamycin), everolimus, mycophenolate mofetil (MMF), azathioprine, prednisone, basiliximab, thymoglobulin, or belatacept. The AVT can be one or more of valganciclovir, ganciclovir, foscarnet, cidofovir, acyclovir, valacyclovir, famciclovir, letermovir, maribavir, or brincidofovir.

Certain embodiments are directed to methods of treating dementia comprising administering mycophenolate mofetil (MMF) to a subject having or at risk of developing a tauopathy, wherein the dose is about 100 mg to 1000 mg. The method can further include co-administering a second immunomodulatory drug (IMD) selected from cyclosporine, tacrolimus, sirolimus (rapamycin), everolimus, azathioprine, prednisone, basiliximab, thymoglobulin, or belatacept. The method can further include co-administering an antiviral therapy (AVT) selected from valganciclovir, ganciclovir, foscarnet, cidofovir, acyclovir, valacyclovir, famciclovir, letermovir, maribavir, or brincidofovir. In certain aspects, MMF, IMD, or AVT is administered at a dose of 0.5 mg to 500 mg daily. In certain aspects, the administration of MMF comprises administering MMF for 10 to 30 days, 2 to 5 weeks, or 1 to 6 months. In certain aspects, MMF can be administered by oral or intranasal administration. Oral administration can be via a tablet, capsule, pill, powder, sustainedrelease formulation, solution, or suspension. In certain aspects, MMF and magnesium (Mg) are co-formulated.

The term “dementia” refers to symptoms affecting memory, thinking, and social abilities severely enough to interfere with daily functioning. Dementia is not a single disease but a syndrome, meaning it is a group of symptoms that can accompany certain diseases or physical conditions. Symptoms include memory loss (particularly affecting recent events or new information), difficulty with language (problems with speaking, understanding, reading, or writing), impaired reasoning and judgment (challenges in planning, organizing, or solving problems), spatial and motor skills (issues with coordination and orientation, leading to getting lost in familiar places), and personality changes (including mood swings, depression, apathy, or becoming socially withdrawn). Dementia typically progresses through stages, from mild (where daily activities can still be managed with some effort) to severe (where complete assistance in daily activities is required). Dementia represents a significant challenge, not just medically but socially and economically, due to its impact on individuals, families, and healthcare systems. Understanding it as a syndrome rather than a single disease helps in addressing the diverse needs of those affected by its various forms.

The terms “treating” or “treatment” refer to any success or indicia of success in the attenuation or amelioration of a pathology or condition, including any objective or subjective parameter such as abatement; remission; diminishing of symptoms; stabilization of symptoms, pathology, or condition, slowing in the rate of degeneration or decline, making the final point of degeneration less debilitating, improving a subject's physical or mental well-being, or prolonging the length of survival. The treatment or amelioration of symptoms can be based on objective or subjective parameters, including the results of a physical examination, neurological examination, and/or psychiatric or cognitive evaluations.

“Effective amount” and “therapeutically effective amount” are used interchangeably herein and refer to an amount of a drug, as described herein, effective to achieve a particular biological or therapeutic result, such as, but not limited to, the biological or therapeutic results disclosed herein.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps), but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Diagram of treatment hypothesis.

FIG. 2. Rate and age distribution of immunomodulatory drug-treated patients with dementia.

FIG. 3. Prevalence of diagnosed AD/dementia in the general population compared to transplant patients treated with MMF-TAC. Patients are grouped according to age at the time of the last follow-up examination or death. The table shows actual numbers in the transplant patient cohort studied. *** p<0.0001 compared to the age-matched group in the general population.

FIG. 4. Percent of AD dementia in women based on treatment received.

FIG. 5. Cumulatively, IMDs (used in transplant and non-transplant settings) had a statistically significant protective effect on AD (AD ICD 9-10 codes alone; HR 0.70; CI 0.65-0.76) as well as on AD-related dementia (ADRD(ICD9-10 codes for AD and related dementia combined; HR 0.85; CI 0.79-0.82).

FIG. 6. Protective effect of antiviral therapy (HR 0.86; CI 0.75-0.99) and metformin (HR 0.85; CI 0.78-0.92).

FIG. 7. Protection with the MMF-TAC combination on both ADRD (HR 0.62; CI 0.50-0.77) and AD (HR 0.48; CI 0.39-0.59) compared to no IMD and to the effect of other IMDs (ADRD HR 0.98; CI 0.91-1.05; and AD HR 0.82; CI 0.76-0.88).

FIG. 8A-8B. (A) Kaplan-Meyer curves showing ADRD-free survival curves when comparing no IMD (none) vs other IMD vs MMF-TAC combination. (B) Cumulative mean function estimate of ADRD by different IMD analyzed by age.

FIG. 9. Triple combination therapy dose adjustment strategy and timeline.

FIG. 10. Study timeline.

FIG. 11. Patient population inclusion criteria.

FIG. 12. Propensity score matching, description of the cohort's demographics.

FIG. 13. Risk ratio distribution of developing Alzheimer's disease, dementia, or cognitive impairments.

FIG. 14. Propensity weighted adjusted hazard of ADRD and AD associated with different IMDs vs no IMD.

FIG. 15A-15B. Survival curves by ADRD and age. (A) Kaplan-Meier curves showing ADRD-free survival curves when comparing no IMD (none) vs other IMD vs MMF-TAC (tacrolimus plus mycophenolate). (B) Cumulative mean function estimate of ADRD by different IMD analyzed by age. The best protection was seen with the drug combination (MMF-TAC). Propensity weighting was used to create both graphs, with the propensity models using all covariates to estimate the probability of treatment for the drug groups. The number at risk represents the propensity-weighted sample size. Bands represent 95% confidence limits for curves. IMD: immunosuppressive drug; MMF: mycophenolate mofetil; TAC: tacrolimus; ADRD: Alzheimer's disease and related dementias.

### DESCRIPTION

The following discussion is directed to various embodiments of the invention. The term “invention” is not intended to refer to any one embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be an example of that embodiment and not intended to imply that the scope of the disclosure, including the claims, is limited to that embodiment.

In AD, evidence of tissue injury, proteolysis, inflammation, and an immune response—manifested by the presence of various cytokines, MHC class I-II antigens, cytotoxic CD8+T cells, B-cells, antibodies, and activated complement-has been demonstrated. Growing evidence supports the notion that AD is a systemic disease, with dysregulation of both the peripheral and central immune systems linked to cognitive dysfunction and clinical status. Emerging studies reveal the critical role of innate immunity pathways in AD pathogenesis, further highlighting their importance. Given the BBB's compromised ability to protect against peripheral and central immune responses in AD, the potential benefits of immunosuppressive treatment become clear.

In this context, expertise in utilizing immunosuppressive drugs (IMD) from clinical organ transplantation is invaluable. Over 30 years of clinical transplant practice have shown that elderly patients receiving IMD after organ transplantation rarely exhibit cognitive decline. A retrospective analysis of 2,644 transplant patients was groundbreaking, demonstrating that the prevalence of AD in these patients is nearly nonexistent (p<0.0001) for age groups over 65, 75, and 85 years old compared to the age-matched general population. These findings were further confirmed through analyses of large datasets of Medicare beneficiaries, revealing that several IMDs are protective in AD, with those used specifically in transplantation offering the best protection. This supports the hypothesis that a crucial immunologic mechanism underlies AD pathology.

### I. Treatment for Dementia, Alzheimer's Disease (ad), and Other Neurodegenerative Disease

Dementia is not a single disease but a term that encompasses a wide range of symptoms associated with a decline in memory or other thinking skills severe enough to reduce a person's ability to perform everyday activities. Different types of dementia include (i) Alzheimer's Disease (AD), the most common cause of dementia, characterized by the buildup of amyloid plaques and tau tangles in the brain. Symptoms include memory loss, difficulty with problem-solving, time and place disorientation, and changes in personality; (ii) Vascular Dementia (VD) occurs due to reduced blood flow to the brain, often following a stroke or series of mini-strokes. VD symptoms can appear suddenly or in a stepwise manner, rather than the gradual onset seen in Alzheimer's. VD can affect cognitive abilities unevenly, with some functions remaining relatively normal while others decline; (iii) Lewy Body Dementia (LBD) characterized by the presence of abnormal protein deposits, called Lewy bodies, in nerve cells. LBD symptoms include visual hallucinations, fluctuations in alertness and attention, motor symptoms similar to Parkinson's disease, and cognitive issues; (iv) Frontotemporal Dementia (FTD) involves the degeneration of the frontal and temporal lobes of the brain. FTD often starts at a younger age than other dementias, typically between 45 and 65. FTD can present with significant changes in personality, behavior, or language difficulties before memory loss becomes apparent; (v) Mixed Dementia (MD) a combination of two or more types of dementia, often Alzheimer's and vascular dementia. MD can complicate diagnosis and treatment because symptoms might not fit neatly into one category; (vi) Parkinson's Disease Dementia (PDD) develops in people with Parkinson's disease, usually years after motor symptoms. PDD cognitive symptoms can include slowed thinking, memory problems, and difficulties with executive function; (vii) Creutzfeldt-Jakob Disease (CJD) a rare, fatal brain disorder caused by prions, leading to rapidly progressive dementia. CJD symptoms can include memory loss, personality changes, and physical problems like coordination issues; (viii) Normal Pressure Hydrocephalus (NPH) caused by the buildup of cerebrospinal fluid in the brain's ventricles, which can be treatable if caught early. NPH symptoms include gait disturbance, urinary incontinence, and dementia-like cognitive impairment; (ix) Huntington's Disease (HD) a genetic disorder leading to the progressive breakdown of nerve cells in the brain. Besides movement disorders, it includes cognitive decline similar to dementia; (x) Wernicke-Korsakoff Syndrome (WKS) often associated with chronic alcoholism due to thiamine (vitamin B1) deficiency. Symptoms include severe memory loss, confusion, and confabulation (making up stories to fill memory gaps).

Each type of dementia can have unique characteristics in terms of symptoms, progression, and impact on daily life. Diagnosis often involves ruling out other conditions and may require a combination of clinical evaluation, imaging (such as MRI or CT scans), and sometimes specific biomarkers or genetic testing. Treatment focuses on managing symptoms, with some types, like NPH, potentially reversible with proper treatment.

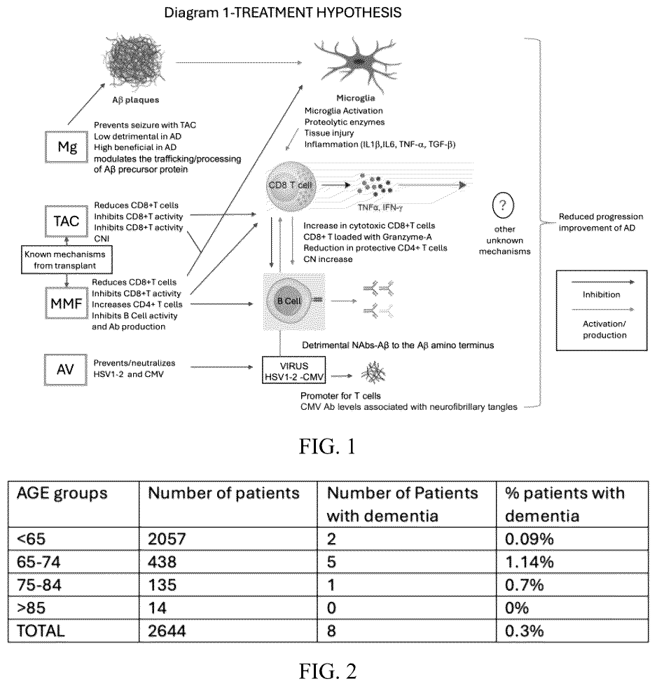

Aspects of the invention are directed to a treatment strategy for dementia and neurodegenerative diseases, such as AD, based on simultaneous pharmacological control of immune activity. In certain aspects, the immune control aims to decrease cytotoxic CD8+T-cells, B-cells, inflammation, calcineurin (CN) hyperactivity, cytokine, and complement activation (all seen with an active immune response) in conjunction with antiviral therapy and magnesium supplementation. Certain aspects are directed to simultaneous treatment with one, two, three, or more of immunomodulatory drugs (IMDs, e.g., MMF-TAC), antiviral therapy (AVT, e.g., valganciclovir), and/or magnesium (Mg) for the purpose of decreasing cognitive decline. An example of the possible targets and mechanisms involved in this treatment hypothesis is schematically summarized in FIG. 1. In certain aspects, the treatment can be directed to other neurodegenerative diseases with (i) an immunologic component, such as multiple sclerosis, amyotrophic lateral sclerosis, autism, Asperger syndrome, Huntington's disease, attention deficit hyperactivity disorder, reward deficiency syndrome, epilepsy, schizophrenia, tic disorders (e.g., Tourette's syndrome), depression, and anxiety, or (ii) a viral component, such as multiple sclerosis, Parkinson's disease, amyotrophic lateral sclerosis, or vascular dementia.

Immunomodulatory drugs (IMD) are commonly used in transplant patients to prevent rejection of the transplanted organ. IMDs include, but are not limited to, (i) cyclosporine, one of the first calcineurin inhibitors used, reduces immune system activity by inhibiting T-cell activation, (ii) tacrolimus, another calcineurin inhibitor, often used as a primary immunosuppressant due to its potency, (iii) sirolimus (rapamycin), an mTOR inhibitor that prevents T-cell proliferation, often used in combination with other drugs or as a replacement for calcineurin inhibitors to reduce nephrotoxicity, (iv) everolimus, an mTOR inhibitor used to prevent rejection and reduce the risk of malignancies, (v) mycophenolate mofetil (MMF)/mycophenolic acid, which inhibits the proliferation of T and B lymphocytes by blocking the synthesis of purines, (vi) azathioprine, an antimetabolite that suppresses the immune system by incorporating into DNA and inhibiting cell division, particularly of lymphocytes, (vii) prednisone/prednisolone, corticosteroids with a broad immunosuppressive effect, reducing inflammation and immune response, (viii) basiliximab, a monoclonal antibody that targets the IL-2 receptor on T-cells, used for induction therapy to delay the onset of rejection, (ix) thymoglobulin (anti-thymocyte globulin), a polyclonal antibody that depletes T-cells, used in induction therapy or for treating acute rejection episodes, (x) belatacept, a fusion protein that inhibits T-cell activation by binding to CD80/86, used as an alternative to calcineurin inhibitors in kidney transplant patients.

MMF-TAC refers to a combination therapy involving mycophenolate mofetil or mycophenolic acid (both indicated as MMF) and tacrolimus (TAC). MMF is an immunosuppressant drug used to prevent rejection in organ transplantation and to treat autoimmune diseases, including lupus nephritis. It works by inhibiting the proliferation of T and B lymphocytes, which are crucial for immune responses. TAC is another immunosuppressant, primarily used to lower the risk of organ rejection after a transplant. It is also employed in treating various autoimmune conditions. Tacrolimus inhibits T-lymphocyte activation, thereby suppressing the immune system. Combination therapy (MMF-TAC) is particularly noted for its use in treating refractory cases of lupus nephritis, where patients do not adequately respond to initial treatments with either MMF or TAC alone.

Transplant patients receiving MMF or MMF-TAC, prophylactic AVT, and magnesium supplementation had almost no prevalence of clinically manifest AD, compared to agematched groups in the general population. Additionally, improvement was observed in a few cases of dementia. Likewise, combined treatment with MMF-TAC was associated with the best protective effect against AD when comparing different IMDs in a larger cohort of transplant patients using Medicare data analysis. This clinical observation suggests that the combination can be a breakthrough treatment to prevent and possibly reverse AD-related cognitive decline. However, this treatment has not been tested in non-transplant patients with AD.

Antiviral therapy (AVT) for use in therapeutic combinations described herein can include, but is not limited to, antiviral medication primarily used for the prevention and treatment of cytomegalovirus (CMV) infections. Representative AVTs include (i) valganciclovir, a prodrug of ganciclovir (ii) ganciclovir (iii) foscarnet (iv) cidofovir (v) acyclovir (vi) valacyclovir (vii) famciclovir (viii) letermovir (ix) maribavir (x) brincidofovir (xi) artemisinin derivatives, and the like.

Magnesium is an essential mineral that plays a crucial role in over 300 enzymatic reactions in the body. It is involved in muscle and nerve function (helping muscles relax and supporting nerve transmission), energy production (essential for ATP synthesis, the body's primary energy source), bone health (contributes to bone formation and influences the activities of calcium), heart health (maintains normal heart rhythm and supports cardiovascular health), and mental health (linked to mood regulation and cognitive functions).

Clinical trials are contemplated to assess the effectiveness of combination treatments in reducing cognitive decline associated with AD. To determine the safety and effectiveness of a combination treatment regimen for halting or reversing cognitive decline in patients diagnosed with mild AD, a controlled, longitudinal, randomized clinical trial in a selected cohort of AD patients can be performed. The effectiveness of combination treatments can be assessed through an array of standardized psychometric tests, neuronal markers, and neuroimaging. These tests will be administered at regular intervals throughout the study. Because of the progressive nature of the disease, the study has been designed to treat patients for at least 2 years. This period will allow the assessment of the efficacy and safety profile of the combination treatment compared to the baseline values obtained at the beginning of the study for each subject. Goals of the trial are to demonstrate that a combination therapy: (1) can be safely used and effectively managed to avoid severe side effects or toxicity; (2) is effective in slowing AD progression; (3) stops progression; and/or (4) induces regression of AD dementia.

Another aspect to be studied includes determining the correlation of combination treatment response with novel markers of neuronal injury and immune system activity. Markers of neurotoxicity (neuron-derived cfDNA and circulating exosomes) and immune system measuring tools (CYLEX, MMDX, and Prospera™) and the association with CD8+T, CD4+T cells, B cells, IL-1β, TNF, IL-1, IL-6, TNF-α, TGF-β, and CN serum levels can be measured to assess if these tests can capture changes during therapy in association with AD symptomatology. Goals are to demonstrate that: (1) immune system function correlates with AD symptomatology (activity increases with worsening and reduces with improvement of cognition) and (2) novel tests in AD are relevant in identifying a pattern of changes that correlate with treatment efficacy.

Studies can assess the central hypothesis that a combined and simultaneous therapeutic approach addressing several known mechanisms of AD neuronal injury and dementia will be safe and effective in reducing (and possibly reversing) the cognitive deficits that clinically characterize AD in humans. Specifically, a clinical study can be conducted to test the effectiveness of a combination of drugs (FDA-approved for transplant or other indications different from AD and with a well-established safety profile) in preventing further cognitive decline and/or ameliorating existing cognitive deficits in a cohort of patients clinically diagnosed with early AD.

MMF, an IMD commonly used in organ transplantation and autoimmune disease, is largely bound to serum albumin. Its mechanism of action involves the production of mycophenolic acid, a potent inhibitor of inosine monophosphate dehydrogenase, the rate-limiting enzyme in the de novo synthesis of guanosine nucleotides. T-and B-lymphocytes are more dependent on this pathway than other cell types. MMF increases beneficial CD4+T cells and blocks the replication of activated T-lymphocytes during the S-phase, thereby favoring the induction of apoptosis. Moreover, depletion of guanosine triphosphate in lymphocytes and monocytes by MMF suppresses the expression of adhesion molecules and the production of nitric oxide by iNOS. Additionally, MMF suppresses primary antibody responses more efficiently than secondary responses. Mycophenolic acid also has synergistic activity with viral DNA polymerase inhibitors and reverse transcription. This antiviral activity could offer additional protection against AD mechanism associated with viral infections. Moreover, it has been shown to have antibacterial and antifungal properties. This drug appears to be an ideal therapeutic choice for AD since it is an immunosuppressive medication with combined T-and B-cell inhibition, as well as anti-inflammatory, antiviral, antibacterial, antifungal, and even anticancer activities, without being nephrotoxic, neurotoxic, or diabetogenic.

Tacrolimus (TAC) is a macrolide antibiotic that binds to T-cell binding protein (FKBP). The TAC-FKBP complex inhibits the phosphatase calcineurin (CN), thus blocking signal transduction and preventing the synthesis of IL-2 and other lymphokines essential to T-lymphocyte function, exerting immunosuppressive activity. This drug appears to be an ideal therapeutic choice for AD since it is a potent immunosuppressive medication, has the capacity to reduce CN activity, and CD8+T cells by inhibiting IL-2, and can easily be delivered to neurons and the CNS.

Valganciclovir is the L-valyl ester of ganciclovir that is rapidly hydrolyzed to ganciclovir after oral administration. It is a potent antiviral therapy (AVT), which suppresses DNA synthesis in infected cells by causing chain termination. It is most effective for CMV, but is also used for other herpes viruses (HSV and VZV) and in immunosuppressed organ transplant patients for prophylaxis as well as therapy of viral infections. Moreover, there is evidence that oral administration of valganciclovir can effectively deliver its active form (ganciclovir) at therapeutic levels in the CNS. This drug appears to be an ideal therapeutic choice for AD since it possesses antiviral activity against viruses that have been associated with AD and can be delivered to the CNS.

Magnesium is a mineral involved in a multitude of biomedical processes in the human body. Its depletion causes marked irritability of the nervous system, resulting in epileptic seizures. In immunosuppressed patients, magnesium is routinely supplemented to maintain blood levels above 1.7 mg/dL to reduce the risk of seizures increased by TAC.

The choice of these representative drugs is based on data showing the greatest efficacy in preventing AD with the MMF-TAC combination. Moreover, in a transplant patient population, where these protective effects were demonstrated, patients also receive antiviral prophylaxis and magnesium supplementation routinely. Proposed studies represent the first critical step in developing a combination therapy as a viable treatment for AD. The likelihood of rapid translation into the AD clinical arena is further supported by the fact that MMF, TAC, valganciclovir, and magnesium are FDA-approved drugs used for many years in therapy and prophylaxis following solid organ transplant in humans, as well as in non-transplant patients for other indications.

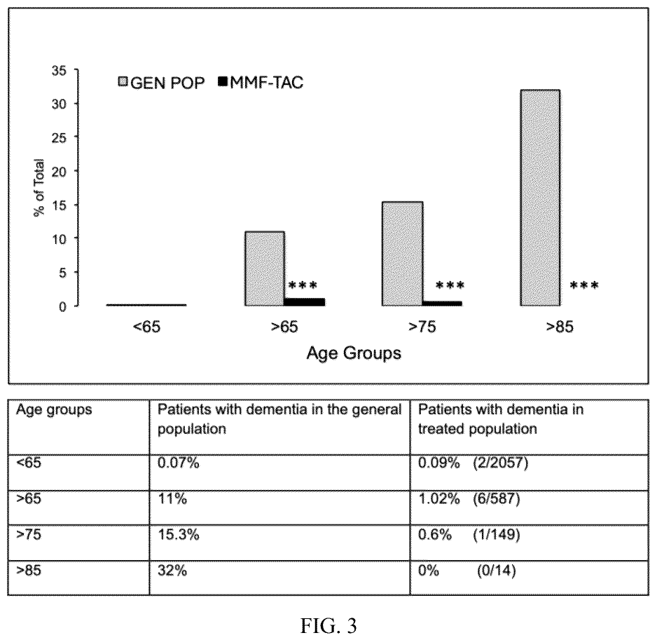

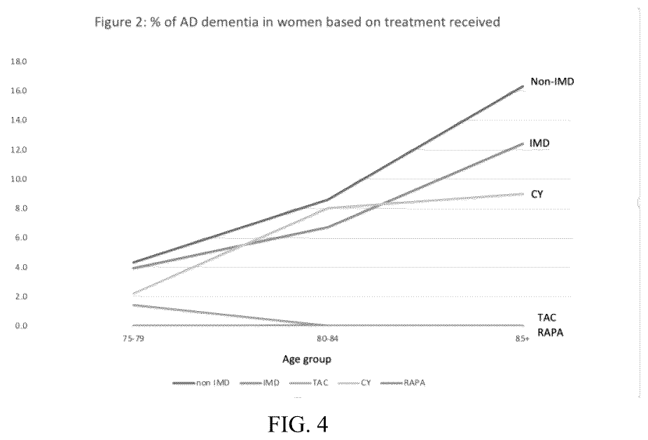

From clinical experience and observation over 30 years of clinical transplantation, it is very uncommon to see transplant recipients with chronic immunosuppression develop dementia in older age groups. A total of 2,644 patients who received 3,167 solid organ transplants were analyzed. These transplant recipients were maintained on MMF-TAC immunosuppressive therapy to prevent allograft rejection. Furthermore, these patients received antiviral prophylaxis and magnesium supplementation. Periodic visits were performed, and medical conditions, including mental status changes, were evaluated and recorded. A retrospective review was performed of the database, evaluating treatment with these drugs, age at the time of analysis, and age at the time of death. The patients were stratified by age groups at the time of the last visit or death: <65; 65-74; 75-84;>85. All patients with altered mental status or any neurologic disorder, including AD, memory loss, or dementia, were identified (FIG. 2). Evidence of dementia was observed in 2 subjects (2/2,057; 0.09%) in the cohort <65 years old, while in the cohort >65, 6 were identified (6/587; 1.02%). Sixty percent of the total patients were male and 40% female, with 6 males and 2 females showing signs of dementia. Notably, 4 of these patients, 1 in the <65 and 3 in the 65-74 age groups, had no mention of dementia in follow-up visits years later (average 5.25 years; range from 1 to 7 years after initial diagnosis) while they were receiving MMF-TAC and steroid-free immunosuppression. It is tempting to speculate that dementia in these patients was haltedor reversed by the treatment, although this has not been substantiated.

National data obtained from the 2014 Alzheimer's Association Facts and Figures dataset were assessed. Comparing similar age groups, a remarkable difference was observed (FIG. 3). These data clearly show that the prevalence of dementia and AD in the patient cohort was nearly absent compared to national data from the general population (*** p<0.0001 for each age group).

These findings are remarkable since several diseases leading to kidney transplantation (by far the most common organ transplant in the general patient population) such as diabetes and hypertension, are also well-established risk factors for dementia and AD. Consequently, it would be expected that the transplant patient population (more than 80% of whom are kidney transplant patients) would be skewed toward a higher rate of developing dementia due to these risk factors. This consideration further supports the predicted protective effect of MMF-TAC treatment in this population. Analysis of 2011-2013, 5% of National Medicare data was conducted, comparing patients not receiving any immunosuppression (non-IMD, n=660,986) to those receiving several IMDs (n=466,905) including TAC, cyclosporine (CY), and rapamycin (RAPA). At the time, the investigation was the impact of calcineurin inhibitors (CNIs) and, for comparative purposes, different IMDs (CNI and non-CNI) were analyzed. Surprisingly, it was discovered that the protective effect was observed not only with CNIs (TAC and, to a lesser extent, CY) but also with a completely different immunosuppressive drug, rapamycin (RAPA) and other IMD medications (FIG. 4).

Rapamycin (RAPA) is an mTOR inhibitor and an IMD with a mechanism of action entirely distinct from that of TAC that does not involve calcineurin inhibition. Additionally, the protective effect seen on AD was more pronounced in women compared to men. There is no difference in calcineurin activity between men and women. Nevertheless, women have a greater susceptibility to AD and autoimmune diseases, and this observed protective effect provides further evidence that immunosuppression is the underlying mechanism. This is additionally substantiated by recent published data that progressively establishes a stronger critical role for CD8+T cells and other immunologic reactions (described above) seen in AD, which closely resemble the immune response observed following organ transplantation.

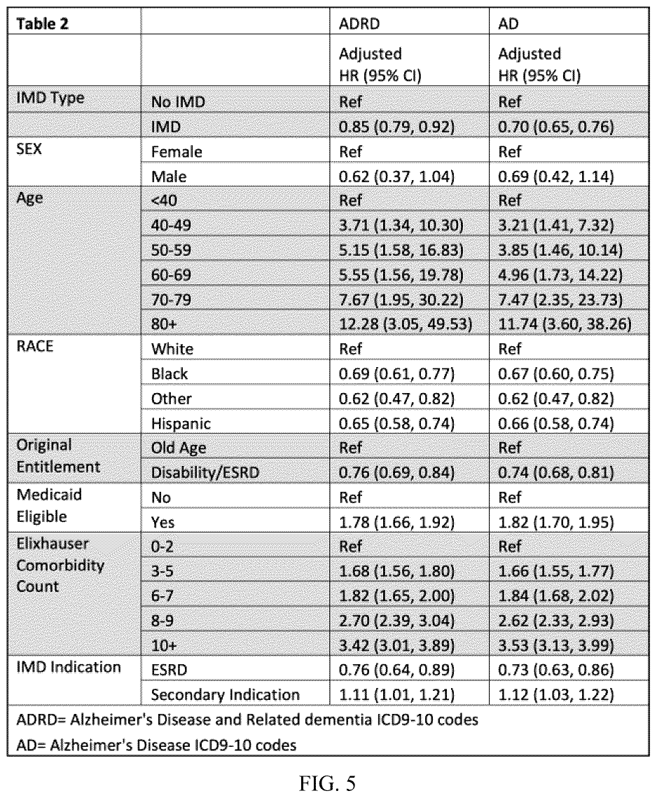

Subsequently, a more granular and focused analysis of transplant patients was conducted utilizing 100% Texas Medicare claims data using the Master Beneficiary Summary File (MBSF), Medicare Provider Analysis and Review (MedPAR) files, Outpatient Standard Analytic (OutSAF) files, Medicare Carrier files, and Part D Event (PDE) files. Beneficiary characteristics were extracted from the MBSF; information regarding diagnosis and procedure was taken from the MedPARand outpatient carrier files, and prescription drug information was extracted from the PDE files. Beneficiaries receiving organ transplant between 2008-2018 (n=19,482) receiving different IMDs (TAC, CY, RAPA, MMF) were compared to patients receiving either no IMD (n=58,119) or other IMDs (abatacept, adalimumab, alefacept, anakinra, azathioprine, basiliximab, belatacept, canakinumab, certolizumab, daclizumab, etanercept, golimumab, infliximab, leflunomide, muromonab-CD3, omalizumab, rilonacept, teriflunomide, tocilizumab, tofacitinib, ustekinumab, and vedolizumab; total n=19,546) matched by age, sex, and race, and stratified by age group. Receipt of IMD was ascertained by filled prescriptions using national drug codes (NDC) or, for injections, using Healthcare Common Procedure Coding System (HCPCS) codes. To account for the potential for changing IMDs, drug type was measured daily following organ transplant. This study confirmed that the protective effect seen on AD is due to immunosuppression. Cumulatively, IMDs (used in transplant) had a statistically significant protective effect on AD (AD ICD 9-10 codes alone; HR 0.70; CI 0.65-0.76) as well as on AD-related dementia (ADRD) (ICD9-10 codes for AD and related dementia combined; HR 0.85; CI 0.79-0.82), summarized in FIG. 5.

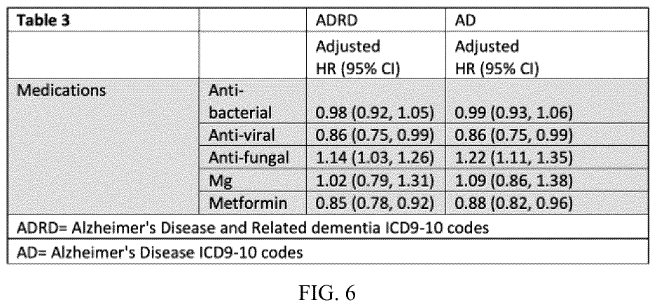

Furthermore, these results demonstrate that this protection is not the result of transplant selection bias. In this analysis, a statistically significant protective effect of antiviral therapy (HR 0.86; CI 0.75-0.99) and metformin (HR 0.85; CI 0.78-0.92) (FIG. 6).

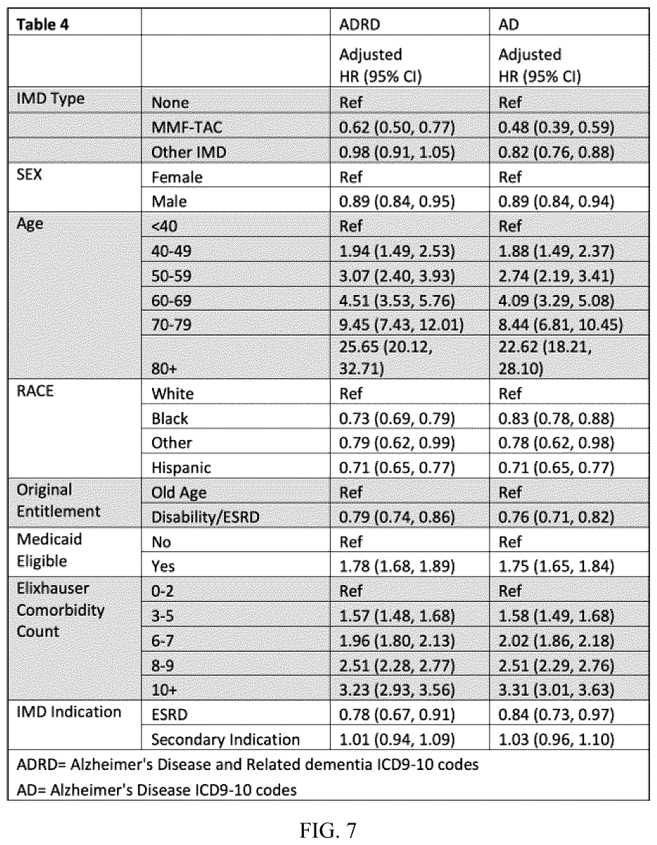

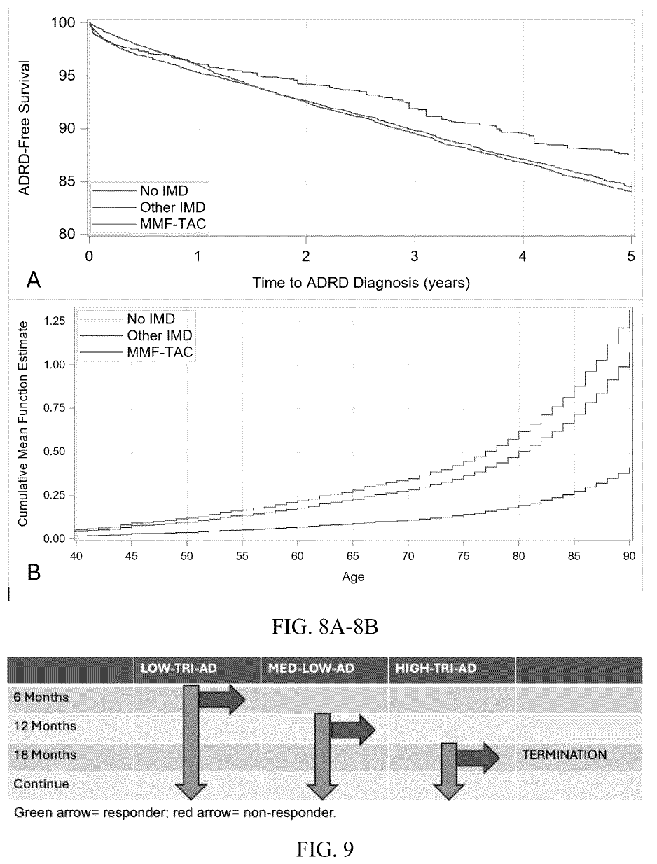

However, the overall best protection was seen with the MMF-TAC combination for both ADRD (HR 0.62; CI 0.50-0.77) and AD (HR 0.48; CI 0.39-0.59) compared to no IMD and to the effect of other IMDs (ADRD HR 0.98; CI 0.91-1.05; and AD HR 0.82; CI 0.76-0.88) (FIG. 7). ADRD-free survival curves (obtained with propensity score analysis) shown in FIG. 8B, additionally support the superior effect of MMF-TAC compared to other IMD and no IMD.

A similar analyisis on this population weighing by inverse probability of treatment weighting (IPTW), variables that continued to be unbalanced were race, ESRD, Elixhauser comorbidity, use of antibacterials, and use of antivirals. These five variables were used as adjustment variables in all subsequent models.

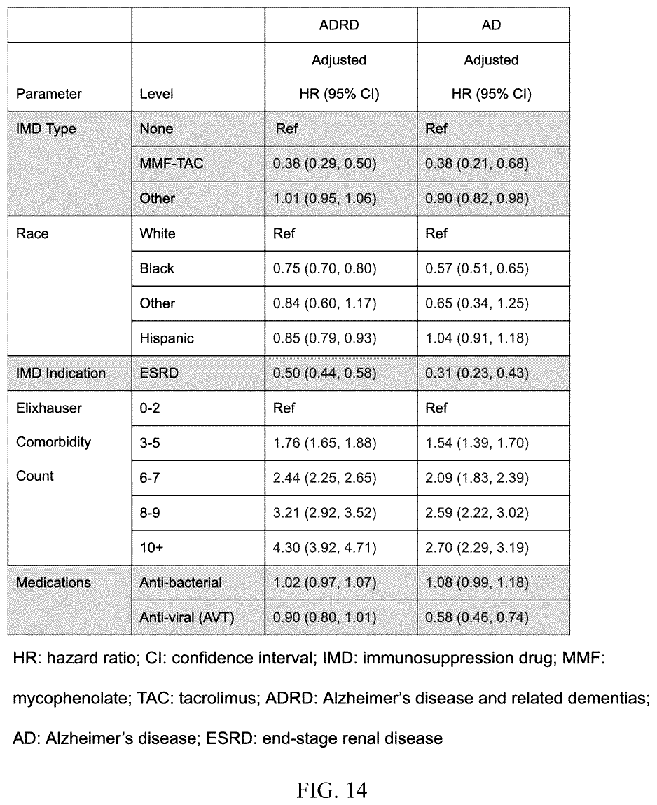

Cumulatively IMD (used in transplant and non) had a statistically significant protective effect on AD (Hazard Ratio [HR]: 0.77; 95% Confidence Interval [CI]:0.69-0.87). Furthermore, these results demonstrate that this protection was not the result of a patient selection bias when listed for organ transplantation. In fact, other IMDs used in non-transplant patients also showed a protective effect. Moreover, a statistically significant protective effect was observed for antiviral therapy AVT (ADRD: HR:0.76; 95%CI:0.63-0.91; AD: HR: 0.55; 95%CI:0.39-0.78). This is shown in FIG. 14.

However, and more importantly, the overall best protection was seen again with the MMF-TAC combination on both ADRD (HR: 0.38; 95%CI:0.29-0.50) and AD (HR: 0.38; 95%CI:0.21-0.68) compared to no IMD (ref), as well as showing a stronger effect than other IMDs (ADRD: HR: 1.01; 95%CI:0.95-1.06; and AD: HR: 0.90; 95%CI:0.82-0.98). This is shown in FIG. 14.

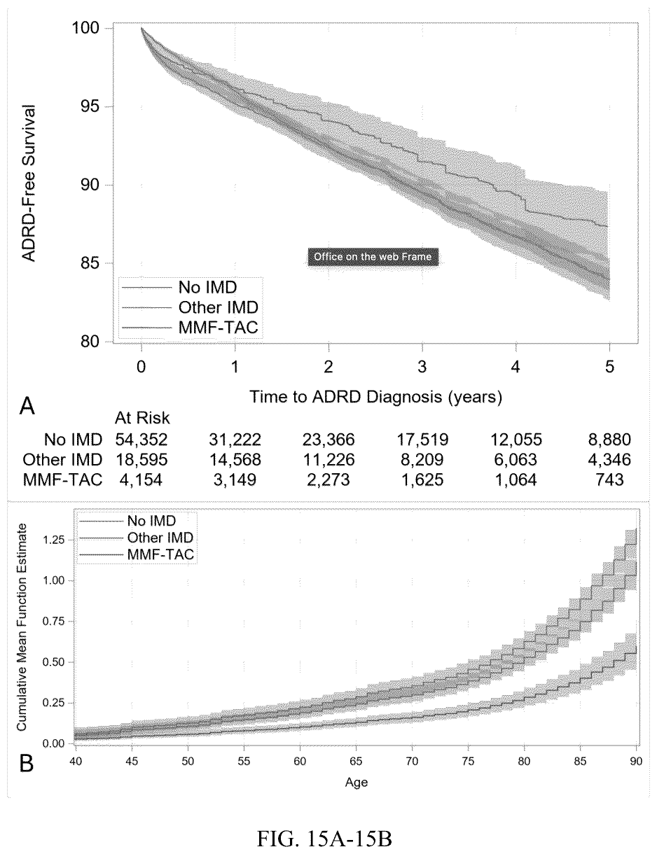

FIG. 15 shows propensity weighted Kaplan Meier survival curves showing ADRD-free survival curves when comparing no IMD (none) vs Other IMD vs MMF-TAC (Tacrolimus plus Mycophenolate). (B) Cumulative mean function estimate of ADRD by different IMD analyzed by age. The best protection was seen with drug combination (MMF-TAC).

This combination is the same used in the transplant patient population, reinforcing that the very low rate of AD observed in patients was not due to the effect of calcineurin inhibitors alone but secondary to the effect of the treatment used in transplant patients, which consists of immunosuppression with MMF-TAC plus other drugs, such as AVT and magnesium. Although magnesium protection in AD showed a trend, it was not statistically significant. However, this electrolyte is clinically necessary when using TAC to increase safety. Moreover, low magnesium levels have been associated with AD deterioration, while high levels are protective. Metformin, which had a far less protective effect than MMF-TAC, is under testing for AD in phase 3 clinical trials. Based on these findings, these drugs were selected for a combination trial. Collectively, compelling preliminary findings substantiate, for the first time in humans, the hypothesis that the treatment used in transplant patients using an IMD drug combination with AVT and magnesium has the best protective effect on AD and maintains the best safety profile. These results highlight the urgent need to test the combination of drugs as a viable novel treatment for AD.

To determine the safety and effectiveness of the TRI-AD treatment regimen (combination of three categories of drugs: IMD, AVT, and magnesium) in halting or reversing cognitive decline in patients diagnosed with mild AD, the following study is proposed: The target is to enroll 150 subjects into the study. Subjects will be randomly assigned to the combination treatment arm (e.g., standard care +MMF +TAC +AVT +magnesium) and a control arm (e.g., standard care +magnesium). Magnesium will be used in both groups. Metformin could also be added to the TRI-AD regimen.

Inclusion Criteria. Subjects (both males and females) will be at least 55 years old, and meet the criteria for AD based on the National Institute of Neurological Disorders and Stroke (NINDS)/Alzheimer's Association criteria and a MiniMental State Examination (MMSE) score of 20-26 at the initial screening visit to be considered mild AD. No restrictions based on level of education or ethnicity will be applied to this initial study, although it is expected that the final study cohort will broadly reflect the ethnic makeup of the catchment area.

Exclusion criteria. To minimize risk for study subjects, exclusion criteria include: age <55 years, lack of adequate family/caregiver support, history of stroke; cancer (<5 years); tuberculosis; chronic obstructive pulmonary disease (COPD); congestive heart failure (CHF); endstage renal disease (ESRD) (estimated by glomerular filtration rate GFR <60); HIV infection; liver disease (hepatitis B, C, or cirrhosis); any active or chronic infection; compromising sensory deficits (vision, hearing, or speech); ongoing anticoagulant treatment; ongoing immunosuppressive therapy.

Protocol. At the initial screening visit, subjects and their legal caregivers will be required to understand and sign a comprehensive IRB-approved informed consent form detailing the study design, procedures, and any risks associated with the test procedures or study drugs. Based on inclusion/exclusion criteria and willingness to participate, subjects will either be enrolled or promptly referred to appropriate specialists for counseling. Enrolled, patients will be administered a comprehensive, standardized cognitive assessment to establish baseline values using the AD Assessment Scale-Cognitive Subscale (ADAS-Cog11) and the AD Cooperative Study-Activities of Daily Living functional scale (ADCS-ADL). They will be required to have up-to-date vaccinations (as per current Infectious Disease recommendations for immunosuppressed patients). Patients will be tested for APOE (ε2-4), HSV1-2, CMV, and EBV. F-18 fluorodeoxyglucose (FDG)-and amyloid-Positron Emission Tomography (PET) scans will be conducted to assess baseline quantitative and spatial distribution of neuronal glucose metabolism and amyloid deposition. At this stage, it is expected to find common FDG patterns in AD with hypometabolism in the temporoparietal medial and lateral cortex, with prominent involvement of the posterior cingulate, precuneus, and posterior parietal cortex. The frontal association cortex is typically spared in early clinical stages. Subjects will then be randomly assigned to the standard care arm (control) or the full treatment arm (standard care plus combination) and assigned a code to protect patient information in data handling pertaining to the study. Subjects in the treatment arm will receive combination therapy for at least six months. Additional study drugs will be provided to the subjects at the time of follow-up visits as necessary. The combination therapy will be dosed and titrated to achieve a target TAC serum level of 3-5 ng/mL. The initial dose will be 1 mg orally once a day and then titrate to reach the desired blood levels. This TAC level is normally low for immunosuppressive maintenance, since levels <3ng/ml are considered non-therapeutic. TAC blood levels are usually kept in transplant patients between 5-10 ng/ml to prevent solid organ rejection in clinical transplant practice. The initial low drug level is used to minimize potential side effects and toxicity, while testing its efficacy. MMF will be dosed using Myfortic (an MMFcoated pill to reduce GI side effects) starting at 360 mg. This is also a low dose, since in transplant patients, a 720 mg dose is typically given. This is reduced to minimize side effects. Valganciclovir and magnesium will be given at the manufacturer's recommended dosage. Therefore, this initial treatment protocol will be low-dose based on the low-dose IMD used. When stable levels are obtained, blood will be drawn monthly unless required by conditions that dictate closer monitoring. Urine samples will also be collected to evaluate urinary tract infections (UTI) and glomerular filtration rate (GFR), if needed. Clinical evaluation will be used to assess possible symptoms of infection or side effects (fever, cough, abdominal pain, diarrhea, constipation). During the first 18 months of the study, subjects will be administered the MMSE, ADASCog11, and ADCS-ADL every three months. After the first 18 months, these tests will be administered every six months. At each of these visits, the subjects will further receive a general well-being assessment, including blood tests for routine vitals (including kidney and liver function) and caregivers will be asked to fill out a questionnaire to assess their experience with the patient's cognitive status.

At each follow-up visit, the study coordinator will compile coded MMSE, ADASCog11, and ADCS-ADL scores and transfer data to the biostatistics team for further analysis. At the end of the first study period (18 months), the principal investigators will assess the cumulative results from MMSE, ADASCog11, and ADCS-ADL scores collected at each of the six 3-month interval follow-up assessments. Then, it is intended to integrate the PET results into the dementia expert's clinical assessment process, disclose the results to the patient, family, and care partners, and engage in discussion regarding the results and their implications for management. In this trial, it is proposed to perform a second set of FDG and amyloid PET exams at the 2-year mark to quantify amyloid burden change and visually evaluate disease pattern or changes.

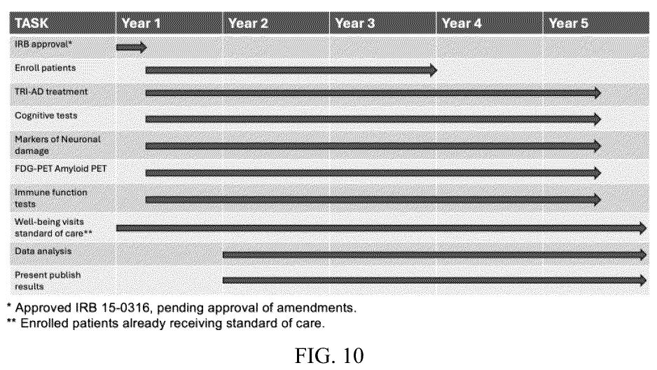

If a beneficial effect of combination treatment is observed at this time, a decision will be made by consensus (incorporating expert input from all investigators) to transition patients from the standard-of-care arm into the treatment arm. Moreover, if after 6 months of the current treatment no significant benefit is achieved, or if a limited benefit is seen in the absence of significant side effects of the low-dose treatment, an increased immunosuppression regimen will be used. This treatment will be called medium immunosuppression treatment and will be achieved using MMF at 580 mg and TAC titrated for a higher blood level (TAC between 5-6 ng/ml). From this time forward, patient progression will be evaluated by comparison with the baseline taken at 18 months. Statistical evaluation methods will be adjusted accordingly. Moreover, if no significant benefit is achieved, or if a limited benefit is seen with no notable adverse effects of the medium-dose treatment after 6 months, a high-dose treatment will be used. This treatment will be called high-dose treatment, and the dose will be adjusted again (MMF 720 mg, while TAC will be titrated to 6-8 ng/ml). These are summarized in FIG. 9. If no beneficial effect is observed after 6 months of high-dose treatment, that patient will be removed from the study to avoid unnecessary risks associated with the treatment and will be considered a non-responder. However, if a patient shows beneficial effects with low or medium dosages without significant side effects, the patient will be moved to the higher dose level to evaluate whether the beneficial effects will further improve. An overall timeline for the project is shown in FIG. 10.

Monitoring of Efficacy of Combination Treatment. In addition to clinical evaluation and cognitive tests, several techniques will be used to measure the effect of combination treatment on the degree of neuronal injury. Neuromarkers such as glial fibrillary acidic protein (GFAP), neurofilament light chain (NFL), plasma phosphorylated tau (p-tau217 and p-tau181); and Aβ42/40 will be periodically tested in the blood. Neuroradiology: On PET scans, standardized uptake value ratio (SUVR) quantitative and Centiloid methods will be used to make regional comparisons of amyloid deposition/distribution from the subject's baseline and computed as the degree of radiotracer uptake in the target region of interest.

Correlation of TRI-AD Treatment Response with Novel Markers of Neuronal Injury and Immune System Activity. Neuron-derived cell-free (cf) DNA circulates through the bloodstream following neuron cell death. A novel test can measure such cfDNA as a means to obtain a liquid biopsy of the neurons to indirectly assess neuronal damage. Therefore, this method will be used before and during combination treatment to assess the degree of neuronal damage in a noninvasive manner and to compare it to clinical findings. Exosomes will also be studied in the peripheral blood before and after combination treatment. Although it is not clear how the immunologic reaction is triggered in AD, it is believed that abnormal antigens (AB, amyloid precursor protein, RNA etc.) are dispersed extracellularly from the damaged neurons and microglia in the peripheral circulation in exosomes and then bind to antigen presenting cells that ultimately provoke an immune reaction resembling the allorecognition seen in transplantation. These exosomes have been described during AD as well as methods for their isolation and identification. Likewise, exosomes are responsible for carrying donor antigens in the process of cross-dressing allorecognition in transplant patients as well as in other diseases. These exosomes will be monitored during the study to assess AD improvement over time from blood and serum). An analysis of the proteins contained in these exosomes will be performed to identify possible immunogenic antigens. Additionally NAbs-Aβ concentration in plasma will be monitored to evaluate what is the correlation between symptoms and immunosuppression.

Immune function monitoring. The degree of immune suppression will be assessed by evaluating immune function using FDA approved methods, such as CYLEX ImmuKnow cell function assay which measures ATP levels of CD4+peripheral T-Lymphocytes and provides an indication of the activity of the immune system. Novel and FDA approved tests developed for donor specific assessment of transplant rejection might be useful in predicting immune status using autologous blood sample of treated patients. Molecular Microscope Diagnostic System (MMDX) uses a GeneChip™ Custom Microarray to measure mRNA transcript levels in biopsies along with extensive big data derived from individuals and populations. The MMDx systems combine these technologies to deliver objective and reproducible transplant biopsy assessments on a molecular level. Prospera™, donor-derived cell-free DNA (dd-cfDNA) technology offers early warning signs of transplant rejection. CD8+T and CD4+T cells, B cells, IL-1β, TNF, IL-1, IL-6, TNF-α TGF-β and CN activity levels will be measured in the peripheral blood using Flow cytometry and cell sorting and ELISA. These circulating cells, cytokines and CN levels will be assessed to determine relevant, and consistent changes to establish characteristic fingerprints during therapy associated with AD symptomatology and treatment response.

Based on transplant clinical experience in elderly patients it is very unlikely that the low dose combination will cause significant side effects or increase risk of developing infections and other conditions associated with lowered immunocompetency. This risk, however, progressively increases with medium and high dose treatments. Therefore, patients in the study group will be monitored with the same modalities that are standard of care for immunosuppressed patients for transplant. Accordingly, patients will be monitored monthly to assess the effect of MMF, TAC blood levels, and evaluate toxicity or side effects. Moreover, if immunologic tests indicate an over-immunosuppressed state, the TRI-AD will be lowered to reduce treatment risk; whereas if immunologic tests indicate an under-immunosuppressed state, the combination treatment will be increased if the patient is a non-responder.

Statistical analysis. At end of the first study period, descriptive statistics and 95% confidence interval for each outcome will be computed for both groups; median and percentiles will be reported for the skewed continuous outcomes. The outcome on the trajectory of MMSE, ADAS-cog11 and ADCS-ADL scores will be assessed with a repeated measure analysis using a linear mixed model approach. Any outcome variables which are positively demonstrated will be log transformed before analysis. Hypothesis tests will be two-sided using the 0.05 significant level. Bonferroni adjustments for multiple tests will be implemented to control for type I errors. Upon completion of the study, we will employ a t-test to compare the change in signal/volume between the two groups, as determined by PET from baseline to study end, provided that the pattern of absent and lost to follow-up remains consistent across all groups. Otherwise, we will adjust patient characteristics with Analysis of Covariance (ANCOVA). For other outcomes, the change from 18 months to the end of the study will be first categorized as a decline of more than 10%, an improvement or more than 10% and between these two levels. We will report and compare descriptive statistics for the categorized outcomes across various patient characteristics. We will further plot the trajectory of these outcomes. Random slope models for these outcomes will be built to assess their change over time and demonstrate whether these changes varied by patients'cognition function at baseline. The slope estimated from the 2nd study period will be compared to the slope estimated from the 1 st study period for patients in the usual care group, as well as for patients in the treatment group. This comparison will help us examine the long-term effect of TRI-AD on cognition impairment. Power analysis: The planned sample size of 75 patients at each group (total sample size: 150) will provide 90% power to detect 80% lower rate of cognition decline in the treatment group versus standard care group at 18 months of follow-up. This sample size calculation is based on a t-test with a 0.05 two-sided significant level and the assumption of average −3.39 MMSE score with standard deviation of 4.59 at 18 months of follow-up in the usual care group [123] and 15% of patients lost in the follow-up period. This sample size will reach 83% power for three-stage sequential testing with the same effect size. All calculations were made using Power Analysis and Sample Size (PASS 2023).

Randomization will assign participants in a 1:1 ratio (Standard of care+Mg or combination). Participants will be randomized via a web-based randomization system in REDCap trial database.

Blinding. The study is a randomized controlled clinical trial. In this particular case we cannot perform a randomized double-blind trial since the dose adjustment of TAC is based on the blood level of the drug and therefore investigators will have knowledge of who is receiving the combination treatment. Moreover, patient receiving the combination treatment being immunosuppressed will also require frequent monitoring of certain clinical parameters and blood tests which are not necessary in patients enrolled in the control arm. Participants enrolled in the study will be blinded to the treatment allocation. Researchers assessing the outcome will be blinded to the allocation of the two treatment groups. Indications are that the patients treated with combination treatment will either have their cognition preserved or will see the rate of decline slowed considerably. It is anticipated that diminished cognitive decline will be accompanied by improved: (1) performance on cognitive tests; (2) on brain imaging with PET-based analyses; (3) serologic neuro-markers of neuronal damage. Immunologic function testing will elucidate the correlation with immunosuppression and efficacy of the combination treatment. Data on humans shows that patients with a beneficial effect on AD had been on these medications for at least one year. However, it is possible that a protective effect will be seen sooner. In our clinical population the treatment significantly prevented AD. Therefore, we have no knowledge of what the effect of the treatment will be on patients who already have developed early AD, and this is the scope of this trial. Data obtained from this trial will provide the base to develop a large scale, multi-center clinical trial of a combination treatment, promoting its ultimate approval by the FDA for clinical use in the treatment and possibly cure of AD.

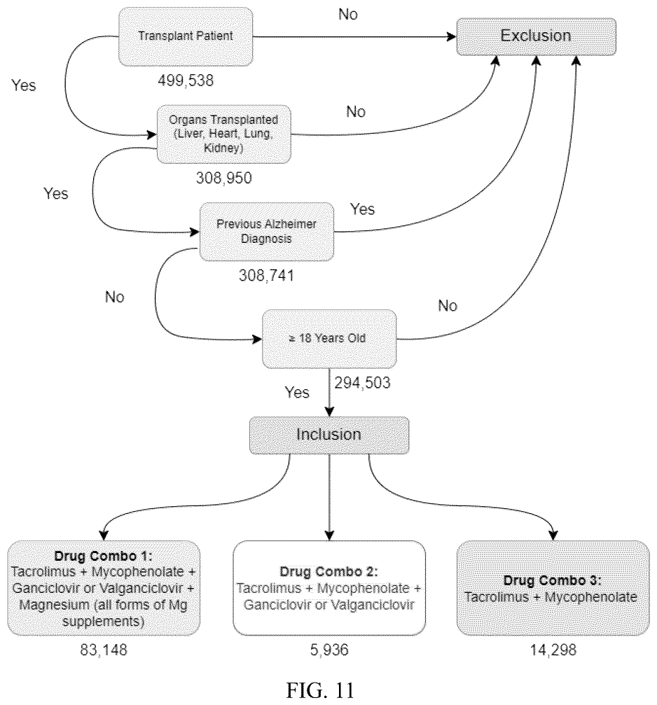

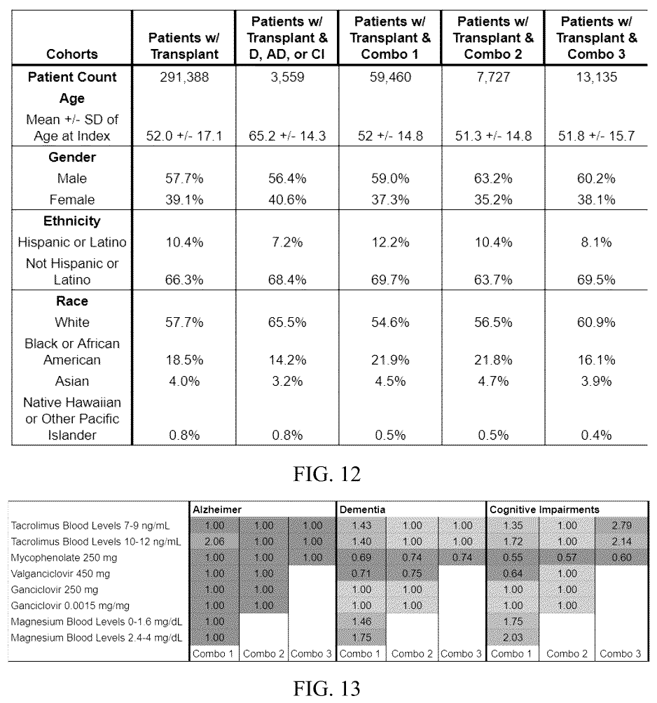

TriNetX, is a global network of electronic medical records from 66 Health care organizations. We used TriNetX to investigate the dose effect of some IMD, AVT and Mg in transplant patients receiving these therapies This cohort included 287,250 adult transplant patients with no history of AD. FIG. 11 illustrates the patient population inclusion criteria when these drugs were divided in 3 combinations (Combo 1 MMF-TAC+AVT+Mg; Combo 2 MMF-TAC+AVT; Combo 3 MMF-TAC). The numbers below each box are the number of patients in each group as we continue to narrow down the pt population. FIG. 12 presents the demographic distribution of patients in these groups.

Risk Ratio (RR) Distribution of Developing Alzheimer Disease and related Dementia and cognitive impairment. A risk ratio is the ratio of how many more cases will occur between one group compared to another. A risk ratio >1 indicates higher risk whereas a risk ratio <1 indicates lower risk. The combinations assessed include Combo 1=Tac+MMF+Antiviral+Magnesium Supplement; Combo 2=Tac+MMF+Antiviral; and Combo 3=Tac+MMF. When comparing these groups to Controls (6,277,004 age-matched patients non receiving any of these drugs), Combo 1 had a RR 0.499; 95%CI 0.34-0.732 in Women and RR 0.612; 95%CI 0.465-0.829 in Men (both p=0.0003). Combo 2 had an overall RR 0.372; 95% CI 0.21-0.659; p=0.0004)

FIG. 13 presents selected results. The number in the box are the risk ratios determined by propensity scores. Therefore, a smaller number indicates a lower risk of developing the associated disorder compared to patients that were not a part of that group.

For TAC, overall, the best protection was seen with blood levels of 7-9 ng/ml. For combo 1, a recommended blood level of 7-9 ng/ml of Tacrolimus to mitigate the risk of Alzheimer's and Cognitive Impairments. However, 10-12 ng/ml of Tac showed a smaller risk of dementia. For combo 2, 7-9 and 10-12 ng/ml of Tac showed the same level of risk for all disorders. For combo 3, we see the same results as combo 2, except 10-12 ng/ml of Tac showed a smaller risk of cognitive impairment.

For combo 1, 2, and 3, 180-250 mg of Mycophenolate is recommended to reduce the risk of all 3 disorders (RR 0.69:95%CI0.578-0.823; p<0.0001).

For combo 1 and 2, Any of the antivirals had the same impact on risk of developing Alzheimer's Disease. However, 450 mg of Valganciclovir showed the largest reduction of risk for cognitive impairments and dementia (RR 0.709; 95% CI0.613-0.82; p<0001).

For Magnesium (only in combo 1), the least increase of risk of all three disorders was observed when the recorded blood level of 0-1.6 mg/dL or 2.4-4.0 mg/dL. Blood levels between 1.6 and 2.4 showed a higher increase in risk of all three disorders.

### II. Pharmaceutical Compositions and Administration Thereof

In light of the current specification, the determination of an appropriate treatment regimen is within the skill of the art. For administration, the components described herein will be formulated in a unit dosage form (solution, suspension, emulsion, etc.) in association with a pharmaceutically acceptable carrier. Such vehicles are usually nontoxic and non-therapeutic. Examples of such vehicles are water, saline, Ringer's solution, dextrose solution, and Hank's solution. Non-aqueous vehicles such as fixed oils and ethyl oleate may also be used. The vehicle may contain minor amounts of additives, such as substances that enhance isotonicity and chemical stability, e.g., buffers and preservatives.

The therapeutic compositions described herein, as well as their biological equivalents, can be administered independently or in combination by any suitable route, oral, nasal spray, or parenteral. Examples of parenteral administration include intravenous, intraarterial, intramuscular, intraperitoneal, and the like. The routes of administration described herein are merely an example and in no way limiting.

The dose of the therapeutic compositions administered to a subject, particularly in a human, in accordance with embodiments of the invention, should be sufficient to result in a desired response in the subject over a reasonable time frame. It is known that the dosage of therapeutic compositions depends upon a variety of factors, including the strength of the particular therapeutic composition employed, the age, species, condition or disease state, and the body weight of the subject. Further disclosed herein, in certain embodiments, is a pharmaceutical composition comprising IMD; AVT; Mg; IMD and AVT; IMD and Mg; AVT and Mg; or IMD, AVT, and Mg. Metformin could also be added to these. In some embodiments, the pharmaceutical composition is in a form suitable for oral administration. In further or additional embodiments, the pharmaceutical composition is in the form of a tablet, capsule, pill, powder, sustained release formulation, solution, or suspension; for parenteral injection as a sterile solution, suspension or emulsion. In further or additional embodiments, the pharmaceutical composition is in unit dosage forms suitable for single administration of precise dosages. In further or additional embodiments, the amount of a compound disclosed herein is in the range of about 0.001 to about 2000 mg/kg body weight/day. In further or additional embodiments, the amount of a compound disclosed herein is in the range of about 0.5 to about 500 mg/kg/day. In further or additional embodiments, the amount of a compound disclosed herein is about 0.5, 25, 50, 100, 200, 300, 400, 500, 600, 700, 800, 900, to about 1000 mg/day.

In certain embodiments a compound(s) disclosed herein is administered in a single dose, once daily. In further or additional embodiments, a compound disclosed herein is administered in multiple doses, more than once per day. In further or additional embodiments, a compound disclosed herein is administered twice daily. In further or additional embodiments, a compound disclosed herein is administered three times per day. In further or additional embodiments, a compound disclosed herein is administered four times per day. In further or additional embodiments, a compound disclosed herein is administered more than four times per day. In some embodiments, the pharmaceutical composition is for administration to a mammal. In further or additional embodiments, the mammal is human. In further or additional embodiments, the pharmaceutical composition further comprises a pharmaceutical carrier, excipient and/or adjuvant. In further or additional embodiments, the pharmaceutical composition comprises a pharmaceutically acceptable salt of a compound disclosed herein.

Disclosed herein, in certain embodiments, is a method for modulating the immune system or immune response of a subject.

Disclosed herein, in certain embodiments, is a method of treating an individual suffering from dementia, a neurodegenerative disease or tauopathy by administering to the individual an effective amount of a composition comprising IMD; AVT; Mg; IMD and AVT; IMD and Mg; AVT and Mg; or IMD, AVT, and Mg as disclosed herein or a pharmaceutically acceptable salts, or prodrugs thereof.

Disclosed herein, in certain embodiments, is a method of treatment of an individual diagnosed with or at risk of developing dementia, or a neurodegenerative disease, e.g., Alzhemier's disease, by administering an effective amount of a composition including IMD; AVT; Mg; IMD and AVT; IMD and Mg; AVT and Mg; or IMD, AVT, and Mg or a pharmaceutically acceptable salts, or prodrugs thereof.

Dose and dosage regimen may depend on the type of biological damage to the host, the type of subject, the history of the subject, and the type of therapeutic composition being administered. The size of the dose will be determined by the route, timing and frequency of administration as well as the existence, nature and extent of any adverse side effects that might accompany the administration of a particular therapeutic composition and the desired physiological effect. It is also known that various conditions or disease states, in particular, chronic conditions or disease states, may require prolonged treatment involving multiple administrations.

Therefore, the amount of the therapeutic composition must be effective to achieve an enhanced therapeutic index. If multiple doses are employed, the frequency of administration will depend, for example, on the type of subject. One skilled in the art can ascertain upon routine experimentation the appropriate route and frequency of administration in a given subject that are most effective in any particular case.

The pharmaceutically acceptable excipients described herein, for example, vehicles, adjuvants, carriers, or diluents, are well known and readily available. It is preferred that the pharmaceutically acceptable carrier be one which is chemically inert with respect to the therapeutic composition and one that has no detrimental side effects or toxicity under the conditions of use.

The choice of excipient will be determined, in part, by the particular therapeutic composition, as well as by the particular method used to administer the composition. Accordingly, there are a wide variety of suitable formulations of the pharmaceutical composition used in the embodiments of the invention. For example, the non-limiting formulations can be injectable formulations such as, but not limited to, those for intravenous, subcutaneous, intramuscular, intraperitoneal injection, and the like, and oral formulations such as, but not limited to, liquid solutions, including suspensions and emulsions, capsules, sachets, tablets, lozenges, and the like. Non-limiting formulations suitable for parenteral administration include aqueous and non-aqueous isotonic sterile injection solutions, including non-active ingredients such as antioxidants, buffers, bacteriostats, solubilizers, thickening agents, stabilizers, preservatives, surfactants, and the like. The solutions can include oils, fatty acids, including detergents and the like, as well as other known and common ingredients in such compositions, without limitation.

1. A method for treating dementia comprising administering a combination therapy comprising a dose of two or more drugs selected from an immunomodulator (IMD), antiviral (AVT), and magnesium (Mg) to a subject having or at risk of developing dementia, wherein the dose of each drug is about 0.5 mg to 1000 mg.

2. The method of claim 1, wherein the subject has or is at risk of developing Alzheimer's disease.

3. The method of claim 1, wherein the IMD is selected, and the IMD is one or more of Cyclosporine, Tacrolimus, Sirolimus (Rapamycin), Everolimus, Mycophenolate Mofetil (MMF), Azathioprine, Prednisone, Basiliximab, Thymoglobulin, or Belatacept.

4. The method of claim 1, wherein the AVT is selected, and the AVT is one or more of Valganciclovir, Ganciclovir, Foscarnet, Cidofovir, Acyclovir, Valacyclovir, Famciclovir, Letermovir, Maribavir, or Brincidofovir.

5. The method of claim 1, wherein the IMD or the AVT is administered at a dose of 0.5mg to 500mg daily.

6. The method of claim 1, wherein the combination therapy is administered for a period selected from 10 to 30 days or 2 to 5 weeks or 1 to 6 months.

7. The method of claim 1, wherein the administering a combination therapy is oral administration or intranasal administration.

8. The method of claim 7, wherein the oral administration comprises a tablet, capsule, pill, powder, sustained-release formulation, solution, or suspension.

9. The method of claim 1, wherein the selected immunomodulator (IMD), the antiviral (AVT), and the magnesium (Mg) are co-formulated.

10. The method of claim 1, wherein the selected immunomodulator (IMD), the antiviral (AVT), and the magnesium (Mg) are administered within 10 to 60 minutes of each other.

11. A composition for treating dementia comprising the co-formulation of an immunomodulator (IMD) and an antiviral (AVT).

12. The composition of claim 11, further comprising magnesium.

13. The composition of claim 11, wherein the IMD is one or more of Cyclosporine, Tacrolimus, Sirolimus (Rapamycin), Everolimus, Mycophenolate Mofetil (MMF), Azathioprine, Prednisone, Basiliximab, Thymoglobulin, or Belatacept.

14. The composition of claim 11, wherein the AVT is one or more of Valganciclovir, Ganciclovir, Foscarnet, Cidofovir, Acyclovir, Valacyclovir, Famciclovir, Letermovir, Maribavir, or Brincidofovir.

15. A method of treating dementia comprising administering Mycophenolate Mofetil (MMF), a first immunomodulatory drug (IMD), to a subject having or at risk of developing a tauopathy, wherein the dose is about 100 mg to 1000 mg.

16. The method of claim 15, further comprising co-administering a second IMD selected from Cyclosporine, Tacrolimus, Sirolimus (Rapamycin), Everolimus, Azathioprine, Prednisone, Basiliximab, Thymoglobulin, or Belatacept.

17. The method of claim 15, further comprising co-administering a second antiviral therapy (AVT) selected from Valganciclovir, Ganciclovir, Foscarnet, Cidofovir, Acyclovir, Valacyclovir, Famciclovir, Letermovir, Maribavir, or Brincidofovir.

18. The method of claim 15, wherein the MMF, the IMD or the AVT is administered at a dose of 0.5mg to 500mg daily.

19. The method of claim 15, wherein the administering MMF comprises administering MMF for 10 to 30 days or 2 to 5 weeks or 1 to 6 months.

20. The method of claim 15, wherein the administering MMF comprises oral administration or intranasal administration.

21. (canceled)

22. (canceled)
